(19) United States
(12) Patent Application Publication
(10) Pub. No.: US 2025/0082196 A1
(43) Pub. Date: Mar. 13, 2025

(54) SELF-PRESCRIBING AUTOMATED PHOROPTER WITH A VIRTUAL OPTOMETRIST

(71) Applicant: iCRx, Inc., Tucson, AZ (US)

(72) Inventors: Erol Ozgur, Tucson, AZ (US); Adoum Mahamat, Tucson, AZ (US)

(21) Appl. No.: 18/463,661

(22) Filed: Sep. 8, 2023

(51) Int. Cl. A61B 3/103; A61B 3/10; G16H 20/00; G16H 80/00

(52) U.S. Cl. CPC A61B 3/103; A61B 3/1015; G16H 20/00; G16H 80/00

(57) ABSTRACT

A self-prescribing auto-phoropter system for digital correction of a patient's eye. The system may comprise a head-mounted optometric device comprising lenses, an audio transceiver, a display component optically in line with the lenses, an optical measurement and correction component for measuring and correcting refractive errors in the eye, and a communication component. The system may further comprise a virtual optometrist device coupled to the communication component, capable of generating a visual eye exam, displaying the visual eye exam to the patient, receiving the one or more refractive errors from the optical measurement and correction component, generating, based on the one or more refractive errors, an eye correction for the patient, applying optometric procedures to optimize the correction, transmitting audio instructions to the patient for the visual eye exam and collecting verbal feedback through the audio transceiver, and generating the final optical prescription for the patient.

### FIELD OF THE INVENTION

The present invention is directed to a self-prescribing automated phoropter that has an AI-based software that performs the routine tasks of an optometrist, which may be defined as a virtual optometrist.

### BACKGROUND OF THE INVENTION

Eye care is an essential part of the medical field, as well as a multi-billion-dollar industry. A significant portion of the world's population requires eye prescriptions for daily use, and this number is growing due to an ever-expanding aging population as well as the increased prevalence of myopia due to the widespread use of advanced visual technologies such as cell phones, television, and laptop or desktop computers.

The World Health Organization anticipates that one-third of the human population will be requiring corrective optics in the next decade. Access to healthcare, both in terms of availability and affordability, will be a serious concern, particularly for low-income communities and developing countries.

Increasing the efficiency of routine optometric exams can help mitigate the difficulties regarding accessing high-quality and affordable eye care. Autophoropters and adaptive optics optometric devices are being used to facilitate eye exams, yet the gold standard in the optometry office is still the subjective refractive testing using a phoropter that relies on verbal feedback from the patient for the doctor to determine the correct prescription. Although there are numerous scientific publications, based on extensive human testing, demonstrating the usefulness of objective refraction, the inability to directly observe a corrected binocular image on an autorefractor steers the optometry specialist to resort to subjective refraction for the best interest of the patient.

Recently, Applicant has developed a prototype device that combines an autorefractor and a phoropter into a single unit. The system can measure the spherical and cylindrical aberrations in the eye objectively with high accuracy and apply an instant correction for any measured refractive errors using a set of tunable fluidic lenses as well as containing integrated visual acuity charts for patient subjective feedback. The device is operated by an optometrist or ophthalmologist, who can perform a measurement and automated correction routine, and fine-tune the prescription if necessary (FIG. 1). The measurement and correction usually take less than five minutes for each patient. Unlike autorefractors, the prescription is final, and no further verification is required. However, due to the requirement of an on-site operator, the instrument is mainly usable at the eyecare facilities only, which requires the patients to schedule appointments and devote a certain amount of time to spend at the facility.

Clearly, eye care technology needs advancement to address the feasibility of providing people with a solution that is more convenient and easier to access. An implementation can be to use an online network of optometrists and/or ophthalmologist that control the test remotely. This enables the placement of the instrument in more convenient places such as shopping malls, where people can get their eyes tested on their time. Moving computation and data management to the Cloud can also help improve the form factor of the instrument and pave the way for a more widespread deployment (FIG. 3).

An indispensable element of instrument-based optometric testing is the operator, who needs to be specialized in testing eyes and prescribing corrections. On the other hand, machine learning algorithms and artificial intelligence (AI) are demonstrated on many occasions to be as capable, if not better, as medical specialists in the diagnosis of many diseases including cancer. Particularly after the COVID-19 pandemic started, virtual medicine quickly emerged and became largely accessible. However, in many conditions, specialized equipment is necessary to perform diagnostic tests, limiting the applicability of virtual medicine, especially eye care among these cases.

An automated optometric system that can perform objective refraction and automated correction can pave the way for eye tests performed by software specifically developed and trained for this task. The present invention is directed to a self-prescribing automated phoropter that has an AI-based software that performs the routine tasks of an operator, a virtual optometrist. The virtual optometrist has the capability to interact with the patients visually and verbally. With this implementation, the automated phoropter becomes self-sufficient to do the optometric exam and provide the patients with a reliable corrective prescription.

### BRIEF SUMMARY OF THE INVENTION

It is an objective of the present invention to provide systems and methods that allow for a self-prescribing automated phoropter that has an AI-based software that performs the routine tasks of a virtual optometrist, as specified in the independent claims. Embodiments of the invention are given in the dependent claims. Embodiments of the present invention can be freely combined with each other if they are not mutually exclusive.

The present invention relates to an automated phoropter that has autorefractive capabilities and tunable lenses that automatically correct for measured refractive errors, all controlled by an artificial intelligence-based virtual optometrist software. The virtual optometrist operates the device by controlling the autorefractive capabilities and automating the given correction by providing the patient with visual and audio cues during the optometric test workflow. The artificial intelligence performs voice recognition by asking the patient to read the exam chart aloud or to verbally confirm the best visual acuity obtained. The virtual optometrist augments the automated phoropter to be a standalone optometric testing station that generates corrective eye prescriptions by combining objective refraction and subjective feedback from the patient.

In state-of-the-art optometric measurement systems, such as autorefractors, or the Opterix™ developed by iCRx, Inc., all the measurements, corrections, and calculations are performed within the unit (FIG. 1). Although it is possible to perform a remote operation using 3rd party remote desktop software, the primary use is on-site.

In another embodiment that was previously disclosed, the calculations and data management tasks previously performed by the instrument were moved to the Cloud as a Cloud-based operation AI. For this system to be operational, a network of remote optometrists and/or ophthalmologists needs to be engaged in the process (FIG. 2).

In the current invention, a virtual optometrist software is introduced into the system. The virtual optometrist software resides in the Cloud, which performs the routine tasks done by the remote optometrist, such as guiding the patient through the optometric test process. Using audio and visual inputs and outputs, the virtual optometrist can engage in patient interaction. Using voice recognition, it listens and deciphers the verbal feedback of the patient at the stage of prescription finalization. Verbal communication can take place in any spoken language that has been programmed. The virtual optometrist software performs the standard procedures for prescription refining such as fogging, cylinder adjustment, etc. The prescription provided by the virtual optometrist is based on objective refraction and automated correction; however, the virtual optometrist has the capability for fine-tuning the prescription through patient feedback (FIG. 4).

In a different embodiment of the invention, the virtual optometrist software is inside the instrument. In this embodiment, limited or no Cloud connection is present, and the instrument has the capability to perform the same tasks in the optometric test workflow with limited or no internet connection (FIG. 5).

One of the unique and inventive technical features of the present invention is the implementation of a virtual optometrist system paired with an in-home phoropter device for instructing the user through use of said device. Without wishing to limit the invention to any theory or mechanism, it is believed that the technical feature of the present invention advantageously provides for a completely automated device for generating an optimal eye correction in a patient and, in some cases, applying said correction directly to the patient. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows the prior automated phoropter system that requires an on-site operator.

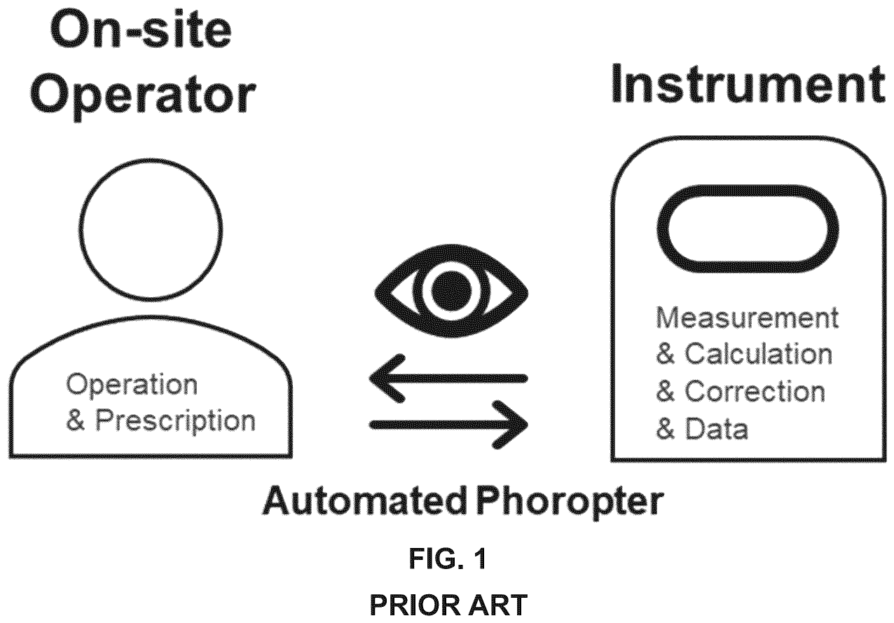

FIG. 2 shows a prior embodiment of the automated phoropter system that can be operated remotely, through a network of remote operators. In this version, the measurement and correction are performed by the device, while the calculations and data management are handled in the cloud. The supervision of an operator is still required.

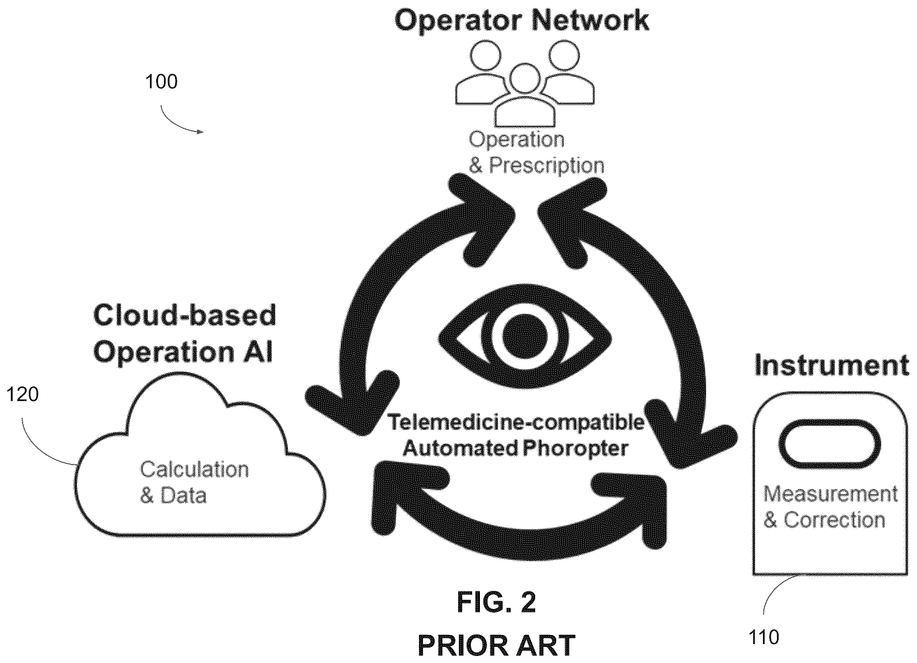

FIG. 3 shows a schematic of an embodiment of the automated phoropter system of the presently claimed invention.

FIG. 4 shows a schematic drawing of an alternate embodiment of the present invention. The virtual optometrist resides in the cloud and performs the standard device operation for autorefraction and automated correction. It has means to visually and verbally interact with the patient mutually. It performs tasks that are being routinely performed by the optometrists, such as confirmation of the objective refraction, and fine-tuning of prescription by applying procedures such as fogging.

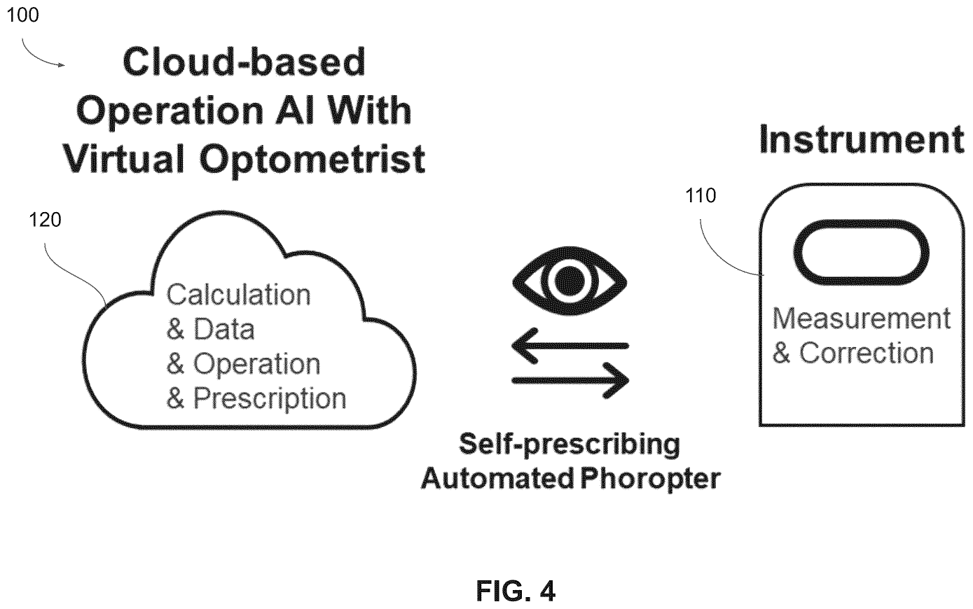

FIG. 5 shows a schematic drawing of another alternative embodiment of the present invention. Here, the virtual optometrist software resides within the instrument, and a Cloud-based operation is either limited or non-existent. The device is mostly or totally self-sufficient. The operational capabilities of the instrument-based virtual optometrist software are equivalent to the Cloud-based virtual optometrist software.

FIG. 6 shows a process diagram of the virtual optometric software of the present invention.

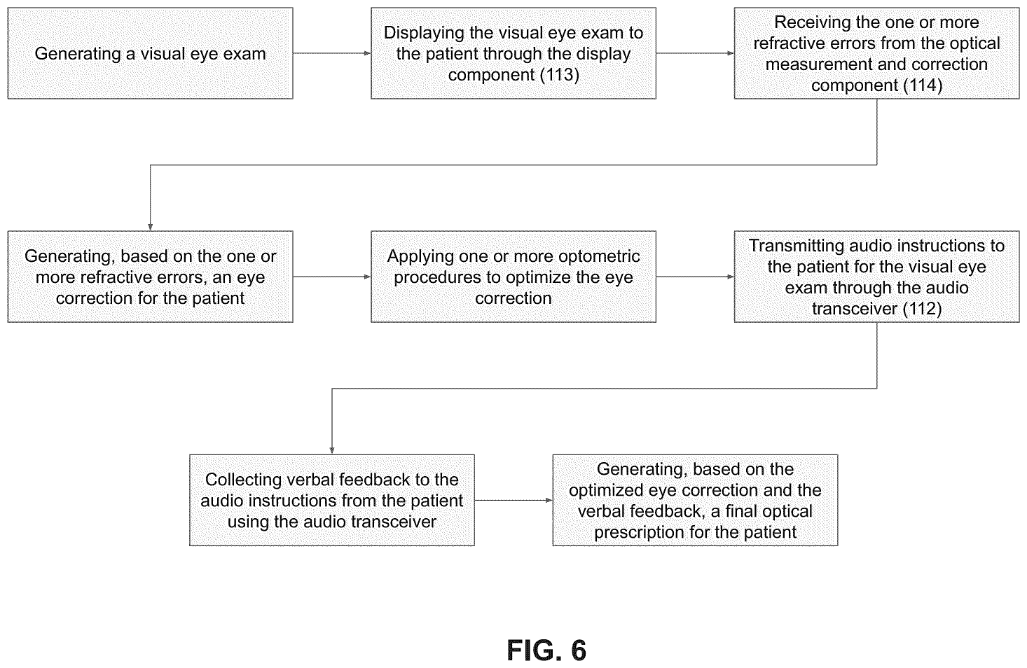

### DETAILED DESCRIPTION OF THE INVENTION

Following is a list of elements corresponding to a particular element referred to herein:
100 self-prescribing auto-phoropter system
110 head-mounted optometric device
111 lenses
112 audio transceiver
113 display component
114 optical measurement and correction component
115 communication component
116 light source
117 mirror
118 beamsplitter
120 virtual optometrist device
121 cloud server

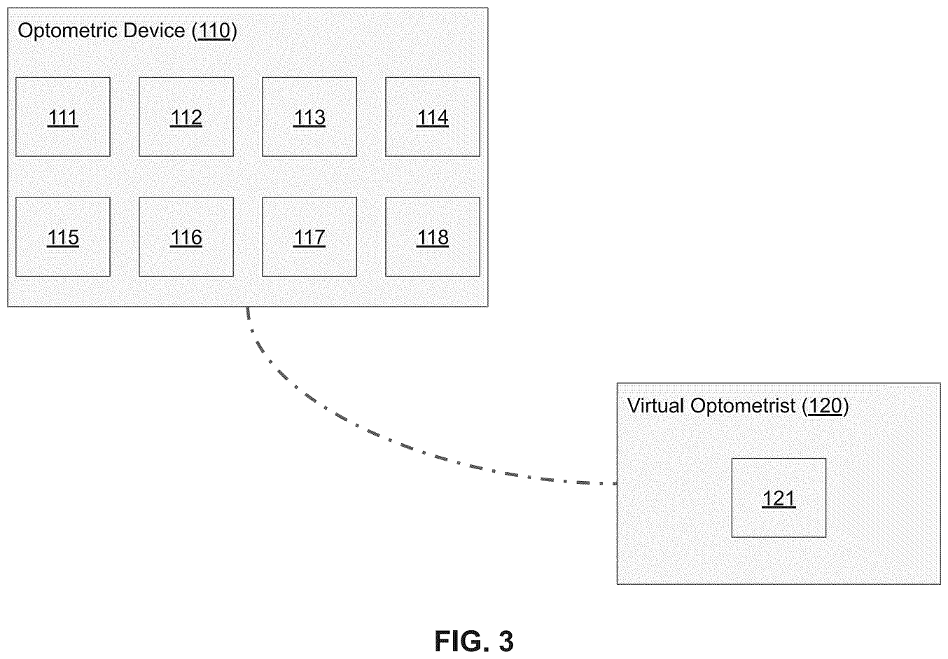

Referring now to FIG. 3, the present invention features a self-prescribing auto-phoropter system (100) for digital correction of an eye of a patient. In some embodiments, the system (100) may comprise a head-mounted optometric device (110). In some embodiments, the head-mounted optometric device (110) may comprise an assembly of tunable lenses (111), an audio transceiver (112) configured to transmit and receive audio data, a display component (113) optically in line with the assembly of tunable lenses (111), configured to display an image, an optical measurement and correction component (114) operatively coupled to the assembly of tunable lenses (111), configured to measure and correct one or more refractive errors in the eye of the patient, and a communication component (115) communicatively coupled to the optical measurement and correction component (114), the audio transceiver (112), and the display component (113).

In some embodiments, the system (100) may further comprise a virtual optometrist device (120) communicatively coupled to the communication component (115) of the head-mounted optometric device (110), comprising a processor capable of executing computer-readable instructions, and a memory component comprising computer-readable instructions. In some embodiments, the computer-readable instructions may comprise generating a visual eye exam, displaying the visual eye exam to the patient through the display component (113), transmitting audio instructions to the patient for the visual eye exam through the audio transceiver (112), receiving verbal responses to the visual eye exam from the patient through the audio transceiver (112), receiving the one or more refractive errors from the optical measurement and correction component (114), and generating, based on the verbal responses and the one or more refractive errors, an optimal eye correction for the patient.

In some embodiments, the optical measurement and correction component (114) may comprise a wavefront sensor. In some embodiments, the wavefront sensor may comprise a Shack-Hartmann sensor. In some embodiments, the wavefront sensor may be configured to measure fixation and visual acuity of the eye of the patient. In some embodiments, the memory component may further comprise instructions for adjusting the optical power of the tunable lenses (111) throughout the visual eye exam. In some embodiments, the memory component may further comprise instructions for applying, by the head-mounted optometric device (110), the optimal eye correction to the eye of the patient. In some embodiments, the visual eye exam may comprise an exam chart, a visual acuity test, or a combination thereof.

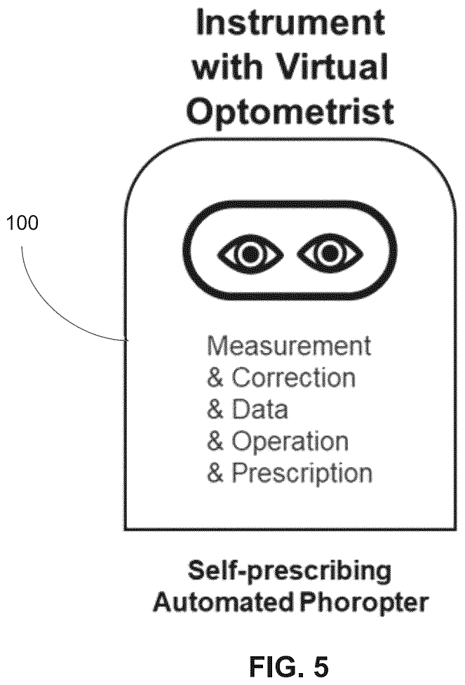

In some embodiments, the virtual optometrist device (120) comprises a cloud server (121), wherein the processor and the memory component may be disposed in the cloud server (121). In these embodiments, the communication component (115) may comprise a communication chip communicatively coupled to the wavefront sensor and the cloud server (121), wherein the communication chip may be configured to transmit the one or more refractive errors and the verbal responses to the cloud server (121). In other embodiments, the virtual optometrist device (120) is local and physically connected to the head-mounted optometric device, as depicted in FIG. 5. In these embodiments, the communication component (115) may comprise a wired connection.

The present invention features a self-prescribing auto-phoropter system (100) for digital correction of an eye of a patient. In some embodiments, the system (100) may comprise a head-mounted optometric device (110). In some embodiments, the head-mounted optometric device (110) may comprise a light source (116) configured to generate light, a mirror (117) configured to reflect light generated by the light source (116) towards a beamsplitter (118), and the beamsplitter (118) configured to split the light into a first beam and a second beam. The first beam may be directed into the eye of the patient, and the second beam may be directed into a wavefront sensor (114). The head-mounted optometric device (110) may further comprise the wavefront sensor (114) configured to compare light reflected from the eye of the patient and the second beam to measure one or more wavefront errors, an audio transceiver (112) configured to transmit and receive audio data, and a assembly of tunable lenses (111) optically in line with a display component (113) and the beamsplitter (118). The first beam may be directed into the eye of the patient through a lens of the assembly of tunable lenses (111). The head-mounted optometric device (110) may further comprise the display component (113) optically in line with the assembly of tunable lenses (111), configured to display an image, and a communication component (115) communicatively coupled to the wavefront sensor (114), the audio transceiver (112), and the display component (113).

In some embodiments, the system (100) may further comprise a virtual optometrist device (120) communicatively coupled to the communication component (115) of the head-mounted optometric device (110). The virtual optometrist (120) may be an AI-based communication software that has been trained to perform an automated optometric exam including objective refraction, refractive correction, and correction optimization using autorefraction data and patient verbal feedback. The virtual optometrist device (120) may comprise a cloud server (121) comprising a processor capable of executing computer-readable instructions, and a memory component comprising computer-readable instructions. In some embodiments, the computer-readable instructions may comprise generating a visual eye exam, displaying the visual eye exam to the patient through the display component (113), transmitting audio instructions to the patient for the visual eye exam through the audio transceiver (112), receiving verbal responses to the audio instructions from the patient through the audio transceiver (112), receiving the one or more wavefront errors from the wavefront sensor (114) throughout the visual eye exam, and generating, based on the verbal responses and the one or more wavefront errors, an optimal eye correction for the patient.

In some embodiments, the computer-readable instructions may comprise generating a visual eye exam, displaying the visual eye exam to the patient through the display component (113), receiving the one or more refractive errors from the optical measurement and correction component (114), generating, based on the one or more refractive errors, an eye correction for the patient, applying one or more optometric procedures to optimize the eye correction, transmitting audio instructions to the patient for the visual eye exam through the audio transceiver (112), collecting verbal feedback to the audio instructions from the patient using the audio transceiver, and generating, based on the optimized eye correction and the verbal feedback, a final optical prescription for the patient.

In some embodiments, the wavefront sensor (114) may comprise a Shack-Hartmann sensor. In some embodiments, the mirror (117) may comprise a dichroic mirror. In some embodiments, the beamsplitter (118) may comprise a polarizing beamsplitter (118). In some embodiments, the system (100) may further comprise a polarizer disposed between the beamsplitter (118) and the wavefront sensor (114). The polarizer may be configured to polarize the second beam and the light reflected from the eye of the patient. In some embodiments, the system (100) may further comprise a communication chip communicatively coupled to the wavefront sensor and the cloud server (121), wherein the communication chip may be configured to transmit the one or more wavefront errors to the cloud server (121).

In some embodiments, the wavefront sensor (114) may be configured to measure fixation and visual acuity of the eye of the patient. In some embodiments, the memory component may further comprise instructions for cycling through the assembly of tunable lenses (111) throughout the visual eye exam. In some embodiments, the memory component may further comprise instructions for applying, by the head-mounted optometric device (110), the optimal eye correction to the eye of the patient. In some embodiments, the visual eye exam may comprise an exam chart, a visual acuity test, or a combination thereof.

In some embodiments, the memory component of the virtual optometrist device (120) may comprise an artificial intelligence (AI) model for natural language processing (NLP). This allows the virtual optometrist device (120) to accept verbal responses to the instructions and the eye exam from the patient through the audio transceiver (112) and process these verbal responses into data to aid in the generation of the optimal eye correction for the patient, along with the refractive errors measured by the optical measurement and correction component (114). In some embodiments, this AI model may be trained using standard language libraries to accept spoken words from the patient as input and generate quantitative data as output.

In some embodiments, each lens of the assembly of tunable lenses may have different properties from the other lenses. In some embodiments, the audio transceiver may comprise any components capable of accepting and playing audio (i.e. a microphone and a speaker). In some embodiments, the display component may comprise any screen capable of displaying digital image data (i.e. a light-emitting diode (LED) screen). In some embodiments, the communication component may comprise a component capable of any form of wireless communication (i.e. a WiFi chip, an antenna configured for wide area network (WAN) connectivity, BlueTooth™ connectivity, etc.), any form of wired communication (e.g. a fiber cable), or a combination thereof.

### EXAMPLE

The following is a non-limiting example of the present invention. It is to be understood that said example is not intended to limit the present invention in any way. Equivalents or substitutes are within the scope of the present invention.

A 35-year-old female comes in with issues with her vision, specifically in focusing on objects at a distance. The patient activates the self-prescribing optometrist system from her home. The system displays a visual eye exam either generated by the system locally or generated in a cloud server and transmitted to the system. The patient views the eye exam through the lens assembly, allowing the wavefront sensor to measure one or more refractive errors of the patient's eyes. Simultaneously, the system will be implementing standard optometric procedures such as fogging and cylinder adjustment to refine the patient's eye correction, and the system will be providing verbal instructions to the patient to walk her through the eye exam. Her verbal responses to these instructions are used to further refine the patient's eye correction. The wavefront errors, the verbal responses, and the optometric procedures are used to generate a final optical prescription for the patient. The patient can then use this automatically generated prescription to get glasses and/or contacts that will aid their near-sightedness.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, MATLAB or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft® Corporation; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

The present invention may comprise or implement a neural network for machine learning tasks. The neural network may be stored, trained, and/or executed entirely on the user's computer, partly on the user's computer, as a stand-alone software package, partly on the user's computer and partly on a remote computer or entirely on the remote computer or server. The neural network may be stored in the form of program code, as described above. The neural network, in some embodiments, may be a perceptron neural network, a feed forward neural network, a multilayer perceptron neural network, a convolutional neural network, a radial basis functional neural network, a recurrent neural network, a long short-term memory neural network, a sequence-to-sequence neural network model, a modular neural network, or the like.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A self-prescribing auto-phoropter system (100) for digital correction of an eye of a patient, the system (100) comprising:
a. a head-mounted optometric device (110) comprising:
i. an assembly of tunable lenses (111);
ii. an audio transceiver (112) configured to transmit and receive audio data;
iii. a display component (113) optically in line with the assembly of tunable lenses (111), configured to display an image;
iv. an optical measurement and correction component (114) operatively coupled to the assembly of tunable lenses (111), configured to measure and correct one or more refractive errors in the eye of the patient; and
v. a communication component (115) communicatively coupled to the optical measurement and correction component (114), the audio transceiver (112), and the display component (113); and
b. a virtual optometrist device (120) communicatively coupled to the communication component (115) of the head-mounted optometric device (110), comprising a processor capable of executing computer-readable instructions, and a memory component comprising computer-readable instructions for:
i. generating a visual eye exam;
ii. displaying the visual eye exam to the patient through the display component (113);
iii. receiving the one or more refractive errors from the optical measurement and correction component (114);
iv. generating, based on the one or more refractive errors, an eye correction for the patient;
v. applying one or more optometric procedures to optimize the eye correction;
vi. transmitting audio instructions to the patient for the visual eye exam through the audio transceiver (112);
vii. collecting verbal feedback to the audio instructions from the patient using the audio transceiver; and
viii. generating, based on the optimized eye correction and the verbal feedback, a final optical prescription for the patient.

2. The system (100) of claim 1, wherein the optical measurement and correction component (114) comprises a wavefront sensor.

3. The system (100) of claim 2, wherein the wavefront sensor comprises a Shack-Hartmann sensor.

4. The system (100) of claim 2, wherein the wavefront sensor is configured to measure fixation and visual acuity of the eye of the patient.

5. The system (100) of claim 1, wherein the memory component further comprises instructions for cycling through the assembly of tunable lenses (111) throughout the visual eye exam.

6. The system (100) of claim 1, wherein the memory component further comprises instructions for applying, by the head-mounted optometric device (110), the optimal eye correction to the eye of the patient.

7. The system (100) of claim 1, wherein the visual eye exam comprises an exam chart, a visual acuity test, or a combination thereof.

8. The system (100) of claim 1, wherein the virtual optometrist device (120) comprises a cloud server (121), wherein the processor and the memory component are disposed in the cloud server (121).

9. The system (100) of claim 8, wherein the communication component (115) comprises a communication chip communicatively coupled to the wavefront sensor and the cloud server (121), wherein the communication chip is configured to transmit the one or more refractive errors and the verbal responses to the cloud server (121).

10. A self-prescribing auto-phoropter system (100) for digital correction of an eye of a patient, the system (100) comprising:
a. a head-mounted optometric device (110) comprising:
i. a light source (116) configured to generate light;
ii. a mirror (117) configured to reflect light generated by the light source (116) towards a beamsplitter (118);
iii. the beamsplitter (118) configured to split the light into a first beam and a second beam, wherein the first beam is directed into the eye of the patient, wherein the second beam is directed into a wavefront sensor (114);
iv. the wavefront sensor (114) configured to compare light reflected from the eye of the patient and the second beam to measure one or more wavefront errors;
v. an audio transceiver (112) configured to transmit and receive audio data;
vi. an assembly of tunable lenses (111) optically in line with a display component (113) and the beamsplitter (118), wherein the first beam is directed into the eye of the patient through a lens of the assembly of tunable lenses (111);
vii. the display component (113) optically in line with the assembly of tunable lenses (111), configured to display an image; and
viii. a communication component (115) communicatively coupled to the wavefront sensor (114), the audio transceiver (112), and the display component (113); and
b. a virtual optometrist device (120) communicatively coupled to the communication component (115) of the head-mounted optometric device (110), comprising a cloud server (121) comprising a processor capable of executing computer-readable instructions, and a memory component comprising computer-readable instructions for:
i. generating a visual eye exam;
ii. displaying the visual eye exam to the patient through the display component (113);
iii. receiving the one or more refractive errors from the optical measurement and correction component (114);
iv. generating, based on the one or more refractive errors, an eye correction for the patient;
v. applying one or more optometric procedures to optimize the eye correction;
vi. transmitting audio instructions to the patient for the visual eye exam through the audio transceiver (112);
vii. collecting verbal feedback to the audio instructions from the patient using the audio transceiver; and
viii. generating, based on the optimized eye correction and the verbal feedback, a final optical prescription for the patient.

11. The system (100) of claim 10, wherein the wavefront sensor (114) comprises a Shack-Hartmann sensor.

12. The system (100) of claim 10, wherein the mirror (117) comprises a dichroic mirror.

13. The system (100) of claim 10, wherein the beamsplitter (118) comprises a polarizing beamsplitter (118).

14. The system (100) of claim 10 further comprising a polarizer disposed between the beamsplitter (118) and the wavefront sensor (114), wherein the polarizer is configured to polarize the second beam and the light reflected from the eye of the patient.

15. The system (100) of claim 10 further comprising a communication chip communicatively coupled to the wavefront sensor and the cloud server (121), wherein the communication chip is configured to transmit the one or more wavefront errors to the cloud server (121).

16. The system (100) of claim 10, wherein the wavefront sensor (114) is configured to measure fixation and visual acuity of the eye of the patient.

17. The system (100) of claim 10, wherein the memory component further comprises instructions for cycling through the assembly of tunable lenses (111) throughout the visual eye exam.

18. The system (100) of claim 10, wherein the memory component further comprises instructions for applying, by the head-mounted optometric device (110), the optimal eye correction to the eye of the patient.

19. The system (100) of claim 10, wherein the visual eye exam comprises an exam chart, a visual acuity test, or a combination thereof.
